(19) United States
(12) Patent Application Publication
(10) Pub. No.: US 2020/0126637 A1
(43) Pub. Date: Apr. 23, 2020

(54) METHODS FOR IDENTIFYING AGENTS WITH DESIRED BIOLOGICAL ACTIVITY

(71) Applicant: The Procter & Gamble Company, Cincinnati, OH (US)

(72) Inventors: Jun Xu, Mason, OH (US); Raghunandan Melkote Kainkaryam, Mason, OH (US)

(21) Appl. No.: 16/720,172

(22) Filed: Dec. 19, 2019

Related U.S. Application Data
(63) Continuation of application No. 15/419,112, filed on Jan. 30, 2017.
Application No. 15/419,112 is a continuation of application No. 13/402,461, filed on Feb. 22, 2012, now abandoned.

(51) Int. Cl. G16B 50/00; G06F 17/16; G16B 20/00; G16B 25/00; G06F 16/2457

(52) U.S. Cl. CPC G16B 50/00; G06F 17/16; G16B 40/00; G16B 25/00; G06F 16/24578; G16B 20/00

(57) ABSTRACT

Provided are methods, systems and apparatus for identifying agents with desired biological activity. Specifically, the methods, systems, and apparatus identify functional relationships between multiple agents and/or between one or more agents and a condition of interest. Data of multiple experimental batches are normalized, batch effects accounted for, and the adjusted data used to create a projection matrix or function. The projection matrix is used to project the data into a projection space, in which the distance between a query agent or a query condition and various candidate agents may be determined.

### BACKGROUND OF THE INVENTION

Connection mapping is a well-known hypothesis generating and testing tool having successful application in the fields of operations research, computer networking and telecommunications. The undertaking and completion of the Human Genome Project and the parallel development of very high throughput, high-density DNA microarray technologies resulted in the generation of an enormous genetic data base. At the same time, the search for new pharmaceutical actives via in silico methods such as molecular modeling and docking studies stimulated the generation of vast libraries of potential small molecule actives. The amount of information linking disease to genetic profile, genetic profile to drugs, and disease to drugs grew exponentially, and application of connectivity mapping as a hypothesis testing tool in the medicinal sciences ripened.

The general notion that functionality could be accurately determined for previously uncharacterized genes, and that potential targets of drug agents could be identified by mapping connections in a data base of gene expression profiles for drug-treated cells, was spearheaded in 2000 with publication of a seminal paper by T. R. Hughes et al. (“Functional discovery via a compendium of expression profiles” Cell 102, 109-126 (2000)), followed shortly thereafter with the launch of The Connectivity Map Project by Justin Lamb and researchers at MIT (“Connectivity Map: Gene Expression Signatures to Connect Small Molecules, Genes, and Disease,” Science, Vol 313 (2006). In 2006, Lamb's group began publishing a detailed synopsis of the mechanics of “C-Map” construction, installments of the reference collection of gene expression profiles used to create the first generation C-Map, and the initiation of an on-going large scale community C-Map project, which is available under the supporting materials hyperlink at http://www.sciencemag.org/content/313/5795/1929/suppl/DCI.

Modern connectivity mapping, with its rigorous mathematical underpinnings and aided by modern computational power, has resulted in confirmed medical successes with identification of new agents for the treatment of various diseases including cancer. Nonetheless certain limiting presumptions challenge application of connectivity mapping with respect to diseases of polyenzymatic origin or syndromic conditions characterized by diverse and often apparently unrelated cellular phenotypic manifestations. According to Lamb, the challenge to constructing a useful connectivity map is in the selection of input reference data which permit generation of clinically salient and useful output upon query. For the drug-related C-Map of Lamb, strong associations comprise the reference associations, and strong associations are the desired output identified as hits. Noting the benefit of high-throughput, high density profiling platforms, Lamb nonetheless cautioned: “[e]ven this much firepower is insufficient to enable the analysis of every one of the estimated 200 different cell types exposed to every known perturbagen at every possible concentration for every possible duration . . . compromises are therefore required” (page 54, column 3, last paragraph). Hence, Lamb confined his C-Map to data from a very small number of established cell lines. Lamb also stressed that particular difficulty is encountered if reference connections are extremely sensitive and at the same time difficult to detect (weak), and Lamb adopted compromises aimed at minimizing numerous, diffuse associations.

A signature-based C-Map query is performed by identifying a list of probe sets corresponding to genes significantly up- or down-regulated in response to, e.g., a condition of interest. This list of probe-sets is called a condition signature. The signature is scored against the C-Map database to identify agents that best replicate or reverse the signature. The signature-based query approach has been used successfully to identify a number of new technologies. However, a condition of interest may involve complex processes involving numerous known and unknown extrinsic and intrinsic factors and responses to such factors may shift over time. This is in contrast to what is typically observed in drug screening methods, wherein a specific target, gene, or mechanism of action is studied. Given the complexity of cellular responses to stimuli, it may be challenging to generate an accurate signature for a biological condition and to distinguish between gene expression data attributable to a perturbagen or condition versus background gene expression data. Thus, for signature-based queries, query signatures should be carefully derived since the predictive value may be dependent upon the quality of the gene signature.

One factor that can impact the quality of a query signature is the number of genes included in the signature. An adequate number of genes must be selected to reflect the dominant and key biology associated with a cellular response to a perturbagen or condition; yet, the set of genes preferably excludes a substantial number of genes exhibiting statistically-significant expression fluctuations due to random chance. With respect to some data architectures and connectivity maps, too few genes (e.g., 500 probe sets out of more than 20,000 measured probe sets) can result in a signature that is unstable with regard to the highest scoring instances; small changes to the query signature can result in significant differences in the highest scoring instance (i.e., small changes in the query signature can significantly alter the query results). The challenges associated with the selection of subsets of probes for signature-based C-Map queries limit the effectiveness of the technology in some instances.

### SUMMARY OF THE INVENTION

The invention provides novel methods, apparatus, and systems useful for identifying agents having a desired biological activity and/or mechanism of action. In particular, the disclosure provides a tool useful for testing and generating hypotheses about agents (i.e., “perturbagens”) and biological conditions based on gene expression data collected over multiple batches. The inventive methods, apparatus, and systems are suitable for, e.g., identifying agents efficacious in the treatment of various conditions.

The present description describes embodiments which broadly include methods, apparatus, and systems for determining relationships between multiple perturbagens. The present description also describes embodiments which broadly include methods, apparatus, and systems for determining relationships between a biological condition of interest and one or more perturbagens. The methods may be used to identify perturbagens impacting the manifestation of a biological condition without detailed knowledge of the biological processes underlying the condition, all of the genes associated with the condition, or the cell types associated with the condition.

A computer-implemented method for constructing a data architecture is stored in a computer-readable storage medium that is communicatively coupled to a processor. The method includes retrieving from a first database of the computer-readable medium a plurality of instances. Each instance corresponds to one of a plurality of batches and includes an expression value for each of a plurality of probes. Each of the plurality of batches results in a plurality of control instances corresponding to gene expression profiles (GEPs) related to controls and a plurality of test instances corresponding to GEPs related to perturbagens. The method also includes selecting from the plurality of probes a subset of probes (which may be all of the probes). The method further includes determining, using the processor, for each batch, an average control GEP. The average control GEP includes only the selected subset of probes and is determined by, for each of the subset of probes, calculating an average expression value for the probe over the plurality of control instances. Additionally, the method includes determining, using the processor, an adjusted GEP for each test instance in a batch. Each adjusted GEP is determined by, for each of the subset of probes, determining the difference between the expression value for the probe in the test instance and the average expression value for the probe in the control instances for the batch. Still further, the method includes storing in a second database of the computer-readable medium a plurality of adjusted instances, each adjusted instance corresponding to one of the adjusted GEPs determined from all of the test instances in all of the plurality of batches.

A data structure includes a matrix of adjusted GEPs. The adjusted GEPs are determined from test instances of a plurality of batches. Each batch includes a plurality of control instances and a plurality of test instances. Each of the adjusted GEPs comprises a difference value, for each of a plurality of probes, between the average expression value for the probe over the plurality of control instances for a particular batch and an expression value for the probe in a test instance within the particular batch.

A method for identifying a candidate perturbagen for treating a condition includes accessing data related to GEP experiments for a plurality of batches. Each batch is associated with a plurality of test instances associated with a perturbagen and a plurality of control instances. Each instance includes an expression value for each of a plurality of probes. The method also includes determining, for each batch, an average control GEP for the batch. The average control GEP is determined by averaging the expression values for each of a subset of probes over all of the control instances. The method further includes determining an adjusted test GEP for each test instance in a batch. Each adjusted GEP is determined by subtracting the expression values for each of the subset of probes in the test instance from the expression value for the corresponding probe in the average control GEP for the corresponding batch. A data matrix is created by combining all of the adjusted test GEPs from all of the plurality of batches. A reduced data matrix is created by removing from the data matrix adjusted test GEPs for any perturbagen for which there exists in the data matrix only a single adjusted test GEP. The method further includes performing a multivariate statistical analysis on the reduced data matrix to create a projection matrix or a projection function defining a projection space, and projecting the data matrix onto the projection space using the projection matrix or the projection function to create a projected matrix. Further, the method includes determining a number of dimensions to keep for the projected matrix (which number may be all of the dimensions). An adjusted condition GEP is determined, and the adjusted condition GEP is projected onto the projection space using the projection matrix or the projection function. The position of the adjusted condition GEP in the projection space is compared to the positions of the adjusted test GEPs in the projection space to identify one or more perturbagens.

In a method for identifying perturbagens having similar biological activity, the method includes accessing data related to GEP experiments for a plurality of batches. Each batch is associated with a plurality of control instances and a plurality of test instances. Each of the plurality of control instances includes information related to a GEP for a control cell and each of the plurality of test instances includes information related to a cell exposed to a corresponding perturbagen. Each of the instances includes an expression value for each of a plurality of probes. The method also includes determining, for each batch, an average control GEP for the batch. The average control GEP for the batch is determined by averaging expression values for each of a subset of probes over all of the control GEPs. The method further includes determining an adjusted test GEP for each test instance in a batch. Each adjusted test GEP is determined by subtracting the expression values for each of the subset of probes in the test instance from the expression value of the average control GEP for the corresponding batch. A data matrix is created by combining all of the adjusted test GEPs from all of the plurality of batches, and a reduced data matrix is created by removing from the data matrix adjusted test GEPs for any perturbagen for which there exists in the data matrix only a single adjusted test GEP. A multivariate statistical analysis is performed on the reduced data matrix to create a projection matrix or a projection function defining a projection space. The data matrix is projected onto the projection space using the projection matrix or the projection function to create a projected matrix. Additionally, the method includes determining a number of dimensions to keep for the projected matrix. The positions of the adjusted test GEPs in the projection space are compared to identify perturbagens with similar biological activity.

A system for identifying candidate perturbagens for treating a condition includes a first database storing a plurality of GEP records. Each GEP record corresponds to one of a plurality of batches and includes, for each of a plurality of GEPs experimentally determined in the batch, an expression value for each of a plurality of probes. Each of the plurality of batches includes a plurality of control GEPs and a plurality of test GEPs. Each of the test GEPs is for a cell exposed to a perturbagen (“a perturbagen GEP”) or a cell exposed to a condition (“a condition GEP”). The system further includes a computer processor communicatively coupled to the database and to a memory device. The memory device stores instructions executable by the processor to retrieve from the first database of the computer-readable medium a plurality of the GEP records. The instructions are further executable to determine, for each batch, an average control GEP for the batch. The average control GEP for the batch includes only a selected subset of probes and is determined by, for each of the subset of probes, calculating an average expression value for the probe over the plurality of control GEPs. The instructions are also executable to determine an adjusted test GEP for each perturbagen GEP in a batch. Each adjusted test GEP is determined by, for each of the subset of probes, determining the difference between the expression value for the probe in the perturbagen GEP and the average expression value for the probe in the control GEP for the corresponding batch. Further, the instructions are executable to create a data matrix by combining all of the adjusted test GEPs from all of the plurality of batches, and to create a reduced data matrix by removing from the data matrix adjusted test GEPs for any perturbagen for which there exists in the data matrix only a single adjusted test GEP. The instructions are executable to perform a multivariate statistical analysis on the reduced data matrix to create a projection matrix or a projection function defining a projection space and to project the data matrix onto the projection space using the projection matrix or the projection function to create a projected matrix. Additionally, the instructions are executable to determine a number of dimensions to keep for the projected matrix, to determine an adjusted condition GEP vector, and to project the adjusted condition GEP vector onto the projection space using the projection matrix or the projection function. The instructions are also executable to compare the position of the adjusted condition GEP in the projection space to the positions of the adjusted test GEPs in the projection space to identify one or more perturbagens.

A system includes a first database storing a plurality of GEP records. Each GEP record corresponds to one of a plurality of batches and includes, for each of a plurality of GEPs experimentally determined in the batch, an expression value for each of a plurality of probes. Each of the plurality of batches includes a plurality of control GEPs and a plurality of perturbagen GEPs. Each of the perturbagen GEPs is for a cell exposed to a perturbagen. The system also includes a computer processor communicatively coupled to the database and to a memory device storing instructions executable by the processor. The instructions are executable to retrieve from the first database of the computer-readable medium a plurality of the GEP records. The instructions are also executable to determine, for each batch, an average control GEP for the batch. The average control GEP for the batch includes only a selected subset of probes and is determined by, for each of the subset of probes, calculating an average expression value for the probe over the plurality of control GEPs. Further, the instructions are executable to determine an adjusted test GEP for each perturbagen GEP in a batch. Each adjusted test GEP is determined by, for each of the subset of probes, determining the difference between the expression value for the probe in the perturbagen GEP and the average expression value for the probe in the control GEP for the corresponding batch. Additionally, the instructions are executable to create a data matrix by combining all of the adjusted test GEPs from all of the plurality of batches and to create a reduced data matrix by removing from the data matrix adjusted test GEPs for any perturbagen for which there exists in the data matrix only a single adjusted test GEP. Still further, the instructions are executable to perform a multivariate statistical analysis on the reduced data matrix to create a projection matrix or a projection function defining a projection space and to project the data matrix onto the projection space using the projection matrix or the projection function to create a projected matrix. The instructions are further executable to determine a number of dimensions to keep for the projected matrix, to receive a selection of an adjusted test GEP corresponding to a query perturbagen; and to compare the position in the projection space of the adjusted test GEP corresponding to the query perturbagen to the positions in the projection space of each of the adjusted test GEPs.

A computer-readable storage medium stores a set of instructions executable by a processor coupled to the computer-readable storage medium. The computer-readable storage medium includes instructions for obtaining data of GEP experiments for a plurality of batches. Each batch results in a plurality of test instances including information related to a perturbagen and a plurality of control instances. Each of the instances includes an expression value for each of a plurality of probes. The storage medium also includes instructions for determining, for each batch, an average control GEP for the batch. The average control GEP for the batch is determined by averaging the expression values for each of a subset of probes over all of the control GEPs. Further, the storage medium includes instructions for determining an adjusted test GEP for each test instance in a batch. Each adjusted test GEP is determined by subtracting the expression values for each of the subset of probes in the test instance from the expression value of the average control GEP for the corresponding batch. Additionally, the storage medium includes instructions for creating a data matrix by combining all of the adjusted test GEPs from all of the plurality of batches and instructions for creating a reduced data matrix by removing from the data matrix adjusted test GEPs for any perturbagen for which there exists in the data matrix only a single adjusted test GEP. Still further, the storage medium includes instructions for performing a multivariate statistical analysis on the reduced data matrix to create a projection matrix or a projection function defining a projection space, instructions for projecting the data matrix onto the projection space using the projection matrix or the projection function to create a projected matrix, and instructions for determining a number of dimensions to keep for the projected matrix. The storage medium also includes instructions for comparing the positions of the adjusted test GEPs in the projection space to identify perturbagens with similar biological activity.

A computer-readable storage medium stores a set of instructions executable by a processor coupled to the computer-readable storage medium. The computer-readable storage medium includes instructions for obtaining data of GEP experiments for a plurality of batches. Each batch results in a plurality of test instances including information related to a perturbagen and a plurality of control instances. Each of the instances includes an expression value for each of a plurality of probes. The storage medium also includes instructions for determining, for each batch, an average control GEP for the batch. The average control GEP for the batch is determined by averaging the expression values for each of a subset of probes over all of the control instances. Further, the storage medium includes instructions for determining an adjusted test GEP for each test instance in a batch. Each adjusted test GEP is determined by subtracting the expression values for each of the subset of probes in the test instance from the expression value of the average control GEP for the corresponding batch. Further still, the storage medium includes instructions for creating a data matrix by combining all of the adjusted test GEPs from all of the plurality of batches, and instructions for creating a reduced data matrix by removing from the data matrix adjusted test GEPs for any perturbagen for which there exists in the data matrix only a single adjusted test GEP. Additionally, the storage medium includes instructions for performing a multivariate statistical analysis on the reduced data matrix to create a projection matrix or a projection function defining a projection space, instructions for projecting the data matrix onto the projection space using the projection matrix or the projection function to create a projected matrix, and instructions for determining a number of dimensions to keep for the projected matrix. The storage medium also includes instructions for determining an adjusted condition GEP, instructions for projecting the adjusted condition GEP onto the projection space using the projection matrix, and instructions for comparing the position of the adjusted condition GEP in the projection space to the positions of the adjusted test GEPs in the projection space to identify one or more perturbagens.

A method for identifying perturbagens having opposite biological activity includes accessing data related to GEP experiments for a plurality of batches. Each batch is associated with a plurality of control instances and a plurality of test instances. Each of the plurality of control instances includes information related to a GEP for a control cell. Each of the plurality of test instances includes information related to a cell exposed to a corresponding perturbagen. Each of the instances includes an expression value for each of a plurality of probes. An average control GEP is determined for each batch. The average control GEP for the batch is determined by averaging expression values for each of a subset of probes over all of the control GEPs. The method further includes determining an adjusted test GEP for each test instance in a batch. Each adjusted test GEP is determined by subtracting the expression values for each of the subset of probes in the test instance from the expression value of the average control GEP for the corresponding batch. A data matrix is created by combining all of the adjusted test GEPs from all of the plurality of batches, and a reduced data matrix is created by removing from the data matrix adjusted test GEPs for any perturbagen for which there exists in the data matrix only a single adjusted test GEP. A multivariate statistical analysis is performed on the reduced data matrix to create a projection matrix or a projection function defining a projection space. The method further includes projecting the data matrix onto the projection space using the projection matrix or the projection function to create a projected matrix, and determining a number of dimensions to keep for the projected matrix. Additionally, the method includes comparing the positions of the adjusted test GEPs in the projection space to identify perturbagens with opposite biological activity.

A method for formulating a composition by identifying similarities between gene expression profiles of cells exposed to different perturbagens includes accessing data related to GEP experiments for a plurality of batches. Each batch is associated with a plurality of control instances and a plurality of test instances. Each of the plurality of control instances includes information related to a GEP for a control cell and each of the plurality of test instances includes information related to a cell exposed to a corresponding perturbagen. Each of the instances includes an expression value for each of a plurality of probes. The method also includes determining, for each batch, an average control GEP for the batch. The average control GEP for the batch is determined by averaging expression values for each of a subset of probes over all of the control GEPs. Further, the method includes determining an adjusted test GEP for each test instance in a batch. Each adjusted test GEP is determined by subtracting the expression values for each of the subset of probes in the test instance from the expression value of the average control GEP for the corresponding batch. A data matrix is created by combining all of the adjusted test GEPs from all of the plurality of batches, and a reduced data matrix is created by removing from the data matrix adjusted test GEPs for any perturbagen for which there exists in the data matrix only a single adjusted test GEP. A multivariate statistical analysis is performed on the reduced data matrix to create a projection matrix or a projection function defining a projection space, and the data matrix is projected onto the projection space using the projection matrix or the projection function to create a projected matrix. The method also includes determining a number of dimensions to keep for the projected matrix, comparing the positions of the adjusted test GEPs in the projection space to identify perturbagens with similar biological activity, and formulating a composition comprising an acceptable carrier and at least one perturbagen selected according to its proximity in the projection space to a second perturbagen.

A method for formulating a composition by identifying differences between gene expression profiles of cells exposed to a perturbagen and gene expression profiles of cells exposed to a condition includes accessing data related to GEP experiments for a plurality of batches. Each batch is associated with a plurality of test instances associated with a perturbagen and a plurality of control instances. Each of the instances includes an expression value for each of a plurality of probes. The method also includes determining, for each batch, an average control GEP for the batch. The average control GEP for the batch is determined by averaging the expression values for each of a subset of probes over all of the control instances. Further, the method includes determining an adjusted test GEP for each test instance in a batch. Each adjusted test GEP is determined by subtracting the expression values for each of the subset of probes in the test instance from the expression value for the corresponding probe in the average control GEP for the corresponding batch. A data matrix is created by combining all of the adjusted test GEPs from all of the plurality of batches and a reduced data matrix is created by removing from the data matrix adjusted test GEPs for any perturbagen for which there exists in the data matrix only a single adjusted test GEP. A multivariate statistical analysis is performed on the reduced data matrix to create a projection matrix or a projection function defining a projection space, and projecting the data matrix onto the projection space using the projection matrix or the projection function to create a projected matrix. Still further, the method includes determining a number of dimensions to keep for the projected matrix, determining an adjusted condition GEP, and projecting the adjusted condition GEP onto the projection space using the projection matrix. Additionally, the method includes comparing the position of the adjusted condition GEP in the projection space to the positions of the adjusted test GEPs in the projection space to identify one or more perturbagens, and formulating a composition comprising an acceptable carrier and at least one perturbagen selected according to the comparison of the positions.

These and additional objects, embodiments, and aspects of the invention will become apparent by reference to the Figures and Detailed Description below.

### BRIEF DESCRIPTION OF THE FIGURES

While the specification concludes with claims particularly pointing out and distinctly claiming the subject matter that is regarded as the invention, it is believed that the invention will be more fully understood from the following description taken in conjunction with the accompanying drawings. Some of the figures may have been simplified by the omission of selected elements for the purpose of more clearly showing other elements. Such omissions of elements in some figures are not necessarily indicative of the presence or absence of particular elements in any of the exemplary embodiments, except as may be explicitly delineated in the corresponding written description. None of the drawings are necessarily to scale.

FIG. 1 is a schematic illustration of a computer system suitable for use with the invention;

FIG. 2 is a schematic illustration of an instance associated with a computer readable medium of the computer system of FIG. 1;

FIG. 3 is a schematic illustration of a programmable computer suitable for use according to the present description;

FIG. 4 is a schematic illustration of an exemplary system for generating an instance;

FIG. 5 depicts a method of identifying similar agents according to the present description;

FIG. 6 depicts a method for identifying candidate agents for treating a condition;

FIG. 7 depicts a method of data preparation in accordance with the methods of FIGS. 5 and 6;

FIG. 8A depicts a method of performing a multivariate statistical analysis in accordance with the methods of FIGS. 5 and 6;

FIG. 8B depicts a method of determining a projection space using regularized Fisher discriminant analysis in a multivariate statistical analysis in accordance with the method of FIG. 8A;

FIG. 9 depicts a method of performing a query for chemical similarity in accordance with the method of FIG. 5;

FIG. 10 depicts a method of performing a query for desired mechanism of action in accordance with the method of FIG. 6;

FIG. 11 depicts a method of selecting probes in accordance with the method of FIG. 7;

FIG. 12 depicts a method of determining an adjusted gene expression profile in accordance with the method of FIG. 7;

FIG. 13 depicts exemplary data structures associated with various embodiments of the present description;

FIG. 14 illustrates exemplary results of a query for agents chemically similar to a query agent;

FIG. 15 illustrates exemplary results related to a query for agents with biological activity similar to a query agent in a first cell line;

FIG. 16 illustrates exemplary results related to a query for agents with biological activity similar to the same query agent in a second cell line; and

FIG. 17 illustrates exemplary results related to a query for agents having gene expression profiles most different from that of a query condition in a cell line.

### DETAILED DESCRIPTION OF THE INVENTION

The invention will now be described with occasional reference to the specific embodiments of the invention. This invention may, however, be embodied in different forms and should not be construed as limited to the embodiments set forth herein. Rather, these embodiments are provided so that this disclosure will be thorough and complete, and to fully convey the scope of the invention to those skilled in the art.

Unless otherwise defined, all technical and scientific terms used herein have the same meaning as commonly understood by one of ordinary skill in the art to which this invention pertains. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise. Unless otherwise indicated, all numerical values are to be understood as being modified in all instances by the term “about.” Additionally, the disclosure of any ranges are to be understood as including the range itself and also anything subsumed therein, as well as endpoints. All numeric ranges are inclusive of narrower ranges; delineated upper and lower range limits are interchangeable to create further ranges not explicitly delineated.

As used herein, the terms “gene expression profiling” and “gene expression profiling experiment” refer to the measurement of the expression of multiple genes in a biological sample using any suitable profiling technology. Exemplary biomolecules representative of gene expression (i.e., “biomarkers”) include protein, nucleic acid (e.g., mRNA or cDNA), protein fragments or metabolites, and/or products of enzymatic activity encoded by the protein encoded by a gene transcript, and detection and/or measurement of any of the biomarkers described herein is suitable in the context of the invention. In one embodiment, the method comprises measuring mRNA encoded by one or more of the genes. If desired, the method comprises reverse transcribing mRNA encoded by one or more of the genes and measuring the corresponding cDNA. Any quantitative nucleic acid assay may be used. For example, many quantitative hybridization, Northern blot, and polymerase chain reaction procedures exist for quantitatively measuring the amount of an mRNA transcript or cDNA in a biological sample. See, e.g., Current Protocols in Molecular Biology, Ausubel et al., eds., John Wiley & Sons (2007), including all supplements. Optionally, the mRNA or cDNA is amplified by polymerase chain reaction (PCR) prior to hybridization. The mRNA or cDNA sample is then examined by, e.g., hybridization with oligonucleotides specific for mRNAs or cDNAs encoded by one or more of the genes of the panel, optionally immobilized on a substrate (e.g., an array or microarray). Selection of one or more suitable probes specific for an mRNA or cDNA, and selection of hybridization or PCR conditions, are within the ordinary skill of scientists who work with nucleic acids. Binding of mRNA or cDNA to oligonucleotide probes specific for the mRNA or cDNA allows for identification and quantification gene expression. For example, the mRNA expression of thousands of genes may be determined using microarray techniques. Other emerging technologies that may be used include RNA-Seq or whole transcriptome sequencing using NextGen sequencing techniques.

As used herein, the term “microarray” refers broadly to any ordered array of nucleic acids, oligonucleotides, proteins, small molecules, large molecules, and/or combinations thereof on a substrate that enables detection and/or quantification of gene expression (i.e., gene expression profiling) in a biological sample. Non-limiting examples of microarrays are available from Affymetrix, Inc.; Agilent Technologies, Inc.; Ilumina, Inc.; GE Healthcare, Inc.; Applied Biosystems, Inc.; and Beckman Coulter, Inc.

The term “perturbagen,” as used herein, means a stimulus used as a challenge in a gene expression profiling experiment to generate gene expression data. Exemplary perturbagens include, but are not limited to, natural products, such as plant or mammal extracts; synthetic chemicals; small molecules; peptides; proteins (such as antibodies or fragments thereof); peptidomimetics; polynucleotides (DNA or RNA); drugs (e.g. Sigma-Aldrich LOPAC (Library of Pharmacologically Active Compounds) collection); and combinations thereof. Other non-limiting examples of perturbagens include botanicals (which may be derived from one or more of a root, stem bark, leaf, seed or fruit of a plant). Some botanicals may be extracted from a plant biomass (e.g., root, stem, bark, leaf, etc.) using one more solvents. A perturbagen composition (e.g., a botanical composition) may comprise a complex mixture of compounds and lack a distinct active ingredient.

By way of example, not limitation, the perturbagen is, in various aspects of the invention, a substance that is Generally Recognized as Safe (GRAS) by the U.S. Food and Drug Administration, a food additive, or a substance used in consumer products including over the counter medications. Some examples of agents suitable for use as perturbagens can be found in: the PubChem database associated with the National Institutes of Health, USA (http://pubchem.ncbi.nlm.nih. gov); the Ingredient Database of the Personal Care Products Council (http://online. personalcarecouncil.org/jsp/Home.jsp); and the 2010 International Cosmetic Ingredient Dictionary and Handbook, 13th Edition, published by The Personal Care Products Council; the EU Cosmetic Ingredients and Substances list; the Japan Cosmetic Ingredients List; the Personal Care Products Council, the SkinDeep database (URL: http://www.cosmeticsdatabase.com); the FDA Approved Excipients List; the FDA OTC List; the Japan Quasi Drug List; the US FDA Everything Added to Food database; EU Food Additive list; Japan Existing Food Additives, Flavor GRAS list; US FDA Select Committee on GRAS Substances; US Household Products Database; the Global New Products Database (GNPD) Personal Care, Health Care, Food/Drink/Pet and Household database (URL: http://www.gnpd.com); and suppliers of cosmetic ingredients and botanicals. In various embodiments, the perturbagen is pathogenic (e.g., a microbe or a virus), radiation, heat, pH, osmotic stress, and the like.

The terms “instance” and “gene expression profile record” as used herein, refer to data related to a gene expression profiling experiment. For example, in some embodiments, the perturbagen is applied to cells, gene expression is detected and/or quantified, and the resulting gene expression data is stored as an instance in a data architecture. The instance may be a “test instance,” which includes gene expression data from cells dosed with a perturbagen; a “condition instance,” which includes gene expression data from cells having a particular phenotype or biological condition under examination (e.g., cells associated with a medical disorder, such as cancer cells, cells affected by rhinovirus infection in a human, or cells infected by a virus or bacterium); or a “control instance” which includes gene expression data from cells not exposed to the perturbagen and not exhibiting a condition of interest (i.e., data from control cells). In some embodiments, the gene expression data comprise a list of identifiers representing the genes that are part of the gene expression profiling experiment. The identifiers may include gene names, gene symbols, microarray probe IDs, or any other identifier. In some embodiments, the gene expression data comprise measurements of gene expression of two or more genes as detected using one or more probes (e.g., oligonucleotide probes). In some embodiments, an instance comprises data from a microarray experiment and includes a list of probe IDs of a microarray ordered by the extent of the differential expression of the probes' target gene(s) relative to gene expression under control conditions. The gene expression data may also comprise metadata, including, but not limited to, data relating to one or more of the perturbagen, the gene expression profiling test conditions, the cells, and the microarray.

As used herein, the term “computer readable medium” refers to any electronic storage medium and includes but is not limited to any volatile, nonvolatile, removable, and non-removable media implemented in any method or technology for storage of information such as computer readable instructions, data and data structures, digital files, software programs and applications, or other digital information. Computer readable media includes, but is not limited to, application specific integrated circuit (ASIC), a compact disk (CD), a digital versatile disk (DVD), a random access memory (RAM), a synchronous RAM (SRAM), a dynamic RAM (DRAM), a synchronous DRAM (SDRAM), double data rate SDRAM (DDR SDRAM), a direct RAM bus RAM (DRRAM), a read only memory (ROM), a programmable read only memory (PROM), an electronically erasable programmable read only memory (EEPROM), a disk, a carrier wave, and a memory stick. Examples of volatile memory include, but are not limited to, random access memory (RAM), synchronous RAM (SRAM), dynamic RAM (DRAM), synchronous DRAM (SDRAM), double data rate SDRAM (DDR SDRAM), and direct RAM bus RAM (DRRAM). Examples of non-volatile memory include, but are not limited to, read only memory (ROM), programmable read only memory (PROM), erasable programmable read only memory (EPROM), and electrically erasable programmable read only memory (EEPROM). A memory can store processes and/or data. Still other computer readable media include any suitable disk media, including but not limited to, magnetic disk drives, floppy disk drives, tape drives, Zip drives, flash memory cards, memory sticks, compact disk ROM (CD-ROM), CD recordable drive (CD-R drive), CD rewriteable drive (CD-RW drive), and digital versatile ROM drive (DVD ROM). As used herein, the term “computer readable storage medium” refers to any computer readable storage medium, excluding carrier waves and other transitory signals.

As used herein, the terms “software” and “software application” refer to one or more computer readable and/or executable instructions that cause a computing device or other electronic device to perform functions, actions, and/or behave in a desired manner. The instructions may be embodied in one or more various forms, such as routines, algorithms, modules, libraries, methods, and/or programs. Software may be implemented in a variety of executable and/or loadable forms and can be located in one computer component and/or distributed between two or more communicating, co-operating, and/or parallel processing computer components and thus can be loaded and/or executed in serial, parallel, and other manners. Software can be stored on one or more computer readable medium and may implement, in whole or part, the methods and functionalities of the invention.

As used herein, the term “data architecture” refers generally to one or more digital data structures comprising an organized collection of data. In some embodiments, the digital data structures can be stored as a digital file (e.g., a spreadsheet file, a text file, a word processing file, a database file, etc.) on a computer readable medium. In some embodiments, the data architecture is provided in the form of a database that may be managed by a database management system (DBMS) that is used to access, organize, and select data (e.g., gene expression profile data) stored in a database. In some embodiments, a database may be stored on a single computer readable medium, while in other embodiments, a database may be stored on and/or across more than one computer readable medium.

### I. Systems and Devices

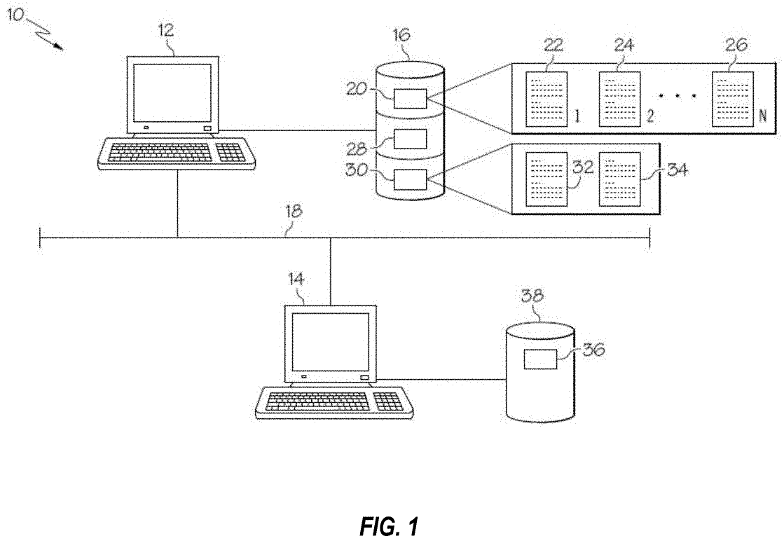

Referring to FIGS. 1, 2, and 4, some examples of systems and devices in accordance with the invention for use in identifying relationships between perturbagens, conditions, and geneswill now be described. System 10 comprises one or more of computing devices 12, 14, a computer readable medium 16 associated with the computing device 12, and communication network 18.

The computer readable medium 16, which may be provided as a hard disk drive, comprises a digital file 20, such as a database file, comprising a plurality of instances 22, 24, and 26 stored in a data structure associated with the digital file 20. The plurality of instances may be stored in relational tables and indexes or in other types of computer readable media. The instances 22, 24, and 26 may also be distributed across a plurality of digital files; a single digital file 20 is exemplified herein merely for simplicity.

The digital file 20 can be provided in wide variety of formats, including but not limited to a word processing file format (e.g., Microsoft Word), a spreadsheet file format (e.g., Microsoft Excel), and a database file format (e.g., GIF, PNG). Some common examples of suitable file formats include, but are not limited to, those associated with file extensions such as *.xls, *.xld, *.xlk, *.xll, *.xlt, *.xlxs, *.dif, *.db, *.dbf, *.accdb, *.mdb, *.mdf, *.cdb, *.fdb, *.csv, *sql, *.xml, *.doc, *.txt, *.rtf, *.log, *.docx, *.ans, *.pages, and *.wps.

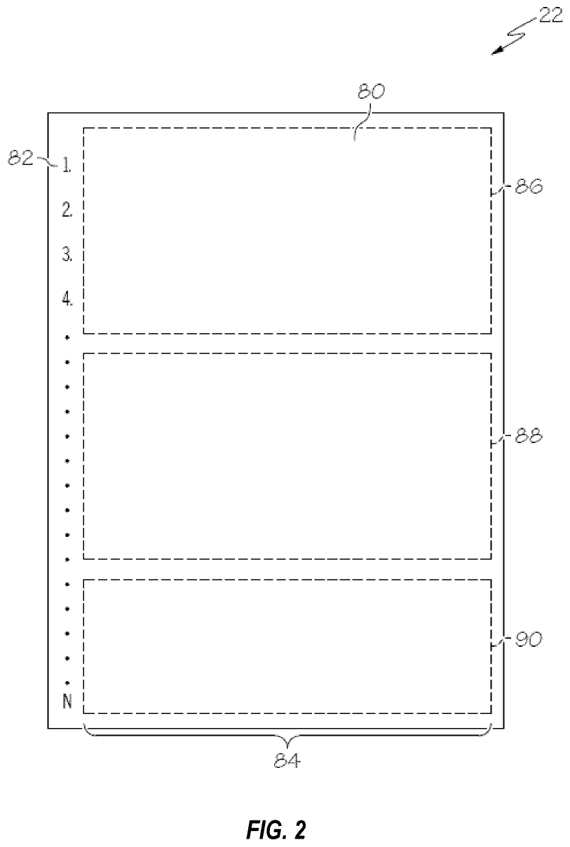

Referring to FIG. 2, in some embodiments the instance 22 may comprise an ordered listing of microarray probe IDs and corresponding expression values, wherein the value of N is equal to the total number of probes on the microarray. Common microarrays include Affymetrix gene chips and Illumina gene chips, both of which comprise probe sets and custom probe sets. Suitable microarray chips include, but are not limited to, those designed for profiling the human genome, such as Affymetrix model Nos. HG-U132 and U133 (e.g., Affymetrix HG-U133APlus2). It will be understood by a person of ordinary skill in the art, however, that any microarray, regardless of proprietary origin, is suitable so long as the probe sets used to construct a data architecture according to the invention are substantially similar.

Instances derived from microarray analyses may comprise an ordered listing of gene probe IDs (and corresponding expression values) where the list comprises, for example, 22,000 or more probe IDs (fewer probe IDs also are contemplated). The ordered listing may be stored in a data structure of the digital file 20 and the data arranged so that, when the digital file is read by the software application 28, a plurality of character strings is reproduced representing the ordered listing of probe IDs. In various embodiments, each instance comprises a full list of the probe IDs, although it is contemplated that one or more of the instances may comprise less than all of the probe IDs of a microarray. It is also contemplated that the instances may include other data in addition to or in place of the ordered listing of probe IDs. For example, an ordered listing of equivalent gene names and/or gene symbols may be substituted for the ordered listing of probe IDs. Additional data may be stored with an instance and/or the digital file 20. In some embodiments, the additional data is referred to as metadata and can include one or more of cell line identification, batch number, exposure duration, and other empirical data, as well as any other descriptive material associated with an instance ID. The ordered list may also comprise a numeric value associated with each identifier that represents the ranked position of that identifier in the ordered list.

Referring again to FIGS. 1, 2, and 3, the computer readable medium 16 may also have a second digital file 30 stored thereon. The second digital file 30 comprises one or more lists 32 of microarray probe IDs associated with one or more conditions. The listing 32 of microarray probe IDs optionally comprises a smaller list of probe IDs than the instances of the first digital file 20. In some embodiments, the list comprises between 2 and 1000 probe IDs. In other specific embodiments the list comprises between 50 and 400 probe IDs. Yet, in some embodiments, the list comprises between 5,000 and 10,000 probe IDs, between 5,000 and 20,000 probe IDs, between 10,000 and 20,000 probe IDs, between 10,000 and 50,000 probe IDs, between 20,000 and 50,000 probe IDs, or all of the probe IDs. The listing 32 of probe IDs of the second digital file 30 comprises a list of probe IDs and corresponding expression values representing up- and/or down-regulated genes selected to represent a condition of interest. In some embodiments, a first list may represent the up-regulated genes and a second list may represent the down-regulated genes of the genetic expression profile. The listing(s) may be stored in a data structure of the digital file 30 and the data arranged so that, when the digital file is read by the software application 28, a plurality of character strings are reproduced representing the list of probe IDs. Instead of probe IDs, equivalent gene names and/or gene symbols (or another nomenclature) may be substituted for a list of probe set IDs. Additional data may be stored with the digital file 30 and this is commonly referred to as metadata, which may include any associated information, for example, cell line or sample source, and microarray identification. In some embodiments, one or more gene expression profiles may be stored in a plurality of digital files and/or stored on a plurality of computer readable media. In other embodiments, a plurality of genetic expression profiles (e.g., 32, 34) may be stored in the same digital file (e.g., 30) or stored in the same digital file or database that comprises the instances 22, 24, and 26.

The data stored in the first and second digital files may be stored in a wide variety of data structures and/or formats, such as the data structures and/or formats described herein. In some embodiments, the data is stored in one or more searchable databases, such as free databases, commercial databases, or a company's internal proprietary database. The database may be provided or structured according to any model, such as, for example and without limitation, a flat model, a hierarchical model, a network model, a relational model, a dimensional model, or an object-oriented model. In some embodiments, at least one searchable database is a proprietary database. A user of the system 10 may use a graphical user interface associated with a database management system to access and retrieve data from the one or more databases or other data sources to which the system is communicatively coupled. In some embodiments, the first digital file 20 is provided in the form of a first database and the second digital file 30 is provided in the form of a second database. In other embodiments, the first and second digital files may be combined and provided in the form of a single file.

In some embodiments, the first digital file 20 may include data that is transmitted across the communication network 18 from a digital file 36 stored on the computer readable medium 38. In one embodiment, the first digital file 20 may comprise gene expression data obtained from a cell line (e.g., a nasal epithelial cell line, a cancer cell line, etc.) as well as data from the digital file 36, such as gene expression data from other cell lines or cell types, perturbagen information, clinical trial data, scientific literature, chemical databases, pharmaceutical databases, and other data and metadata. The digital file 36 may be provided in the form of a database, including but not limited to Sigma-Aldrich LOPAC collection, Broad Institute CMAP collection, GEO collection, and Chemical Abstracts Service (CAS) databases.

The computer readable medium 16 (or another computer readable media, such as 16) may also have stored thereon one or more digital files 28 comprising computer readable instructions or software for reading, writing to, or otherwise managing and/or accessing the digital files 20, 30. The computer readable medium 16 may also comprise software or computer readable and/or executable instructions that cause the computing device 12 to perform one or more methods described herein, including for example and without limitation, methods (or portions of methods) associated with comparing a gene expression profile data stored in digital file 30 to instances 22, 24, and 26 stored in digital file 20, methods (or portions of methods) for comparing gene expression profile data associated with one or more perturbagens, and/or methods (or portions of methods) for comparing (i) gene expression profile data related to a condition to (ii) gene expression profile data related to one or more therapeutic agents. In some embodiments, the one or more digital files 28 form part of a database management system for managing the digital files 20, 28. Non-limiting examples of database management systems are described in U.S. Pat. Nos. 4,967,341 and 5,297,279.

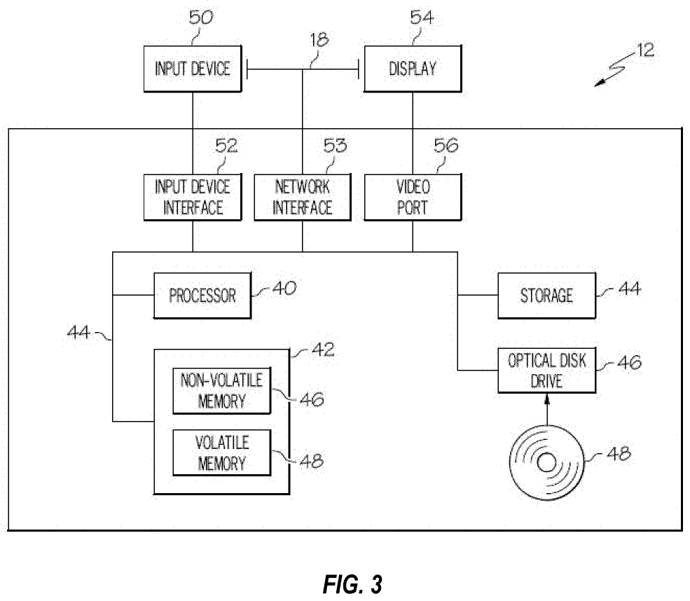

The computer readable medium 16 may form part of or otherwise be connected to the computing device 12. The computing device 12 can be provided in a wide variety of forms, including but not limited to any general or special purpose computer such as a server, a desktop computer, a laptop computer, a tower computer, a microcomputer, a mini computer, a tablet computer, a smart phone, and a mainframe computer. While various computing devices may be suitable for use with the invention, a generic computing device 12 is illustrated in FIG. 3. The computing device 12 may comprise one or more components selected from a processor 40, system memory 42, and a system bus 44. The system bus 44 provides an interface for system components including, but not limited to, the system memory 42 and processor 40. The system bus 36 can be any of several types of bus structures that may further interconnect to a memory bus (with or without a memory controller), a peripheral bus, and a local bus using any of a variety of commercially available bus architectures. Examples of a local bus include an industrial standard architecture (ISA) bus, a microchannel architecture (MSA) bus, an extended ISA (EISA) bus, a peripheral component interconnect (PCI) bus, a universal serial (USB) bus, and a small computer systems interface (SCSI) bus. The processor 40 may be selected from any suitable processor, including but not limited to, dual microprocessor and other multi-processor architectures. The processor executes a set of stored instructions associated with one or more program applications or software.

The system memory 42 can include non-volatile memory 46 (e.g., read only memory (ROM), erasable programmable read only memory (EPROM), electrically erasable programmable read only memory (EEPROM), etc.) and/or volatile memory 48 (e.g., random access memory (RAM)). A basic input/output system (BIOS) can be stored in the non-volatile memory 38, and can include the basic routines that help to transfer information between elements within the computing device 12. The volatile memory 48 can also include a high-speed RAM, such as static RAM for caching data.

The computing device 12 may further include a storage 44, which may comprise, for example, an internal hard disk drive (HDD) (e.g., enhanced integrated drive electronics (EIDE) or serial advanced technology attachment (SATA)) for storage. The computing device 12 may further include an optical disk drive 46 (e.g., for reading a CD-ROM or DVD-ROM 48). The drives and associated computer-readable media provide non-volatile storage of data, data structures and the data architecture of the invention, computer-executable instructions, and so forth. For the computing device 12, the drives and media accommodate the storage of any data in a suitable digital format. Although the description of computer-readable media above refers to an HDD and optical media such as a CD-ROM or DVD-ROM, it should be appreciated by those skilled in the art that other types of media which are readable by a computer, such as Zip disks, magnetic cassettes, flash memory cards, cartridges, and the like may also be used, and further, that any such media may contain computer-executable instructions for performing the inventive methods.

A number of software applications can be stored on the drives 44 and volatile memory 48, including an operating system and one or more software applications, which implement, in whole or part, the functionality and/or methods described herein. It is to be appreciated that the embodiments can be implemented with various commercially available operating systems or combinations of operating systems. The central processing unit 40, in conjunction with the software applications in the volatile memory 48, may serve as a control system for the computing device 12 that is configured to, or adapted to, implement the functionality described herein.

A user may be able to enter commands and information into the computing device 12 through one or more wired or wireless input devices 50, for example, a keyboard, a pointing device, such as a mouse (not illustrated), or a touch screen. These and other input devices are often connected to the central processing unit 40 through an input device interface 52 that is coupled to the system bus 44 but can be connected by other interfaces, such as a parallel port, an IEEE 1394 serial port, a game port, a universal serial bus (USB) port, an IR interface, etc. The computing device 12 may drive a separate or integral display device 54, which may also be connected to the system bus 44 via an interface, such as a video port 56.

The computing devices 12, 14 may operate in a networked environment across network 18 using a wired and/or wireless network communications interface 58. The network interface port 58 can facilitate wired and/or wireless communications. The network interface port can be part of a network interface card, network interface controller (NIC), network adapter, or LAN adapter. The communication network 18 can be a wide area network (WAN) such as the Internet, or a local area network (LAN). The communication network 18 can comprise a fiber optic network, a twisted-pair network, a Tl/El line-based network or other links of the T-carrier/E carrier protocol, or a wireless local area or wide area network (operating through multiple protocols such as ultra-mobile band (UMB), long term evolution (LTE), etc.). Additionally, communication network 18 can comprise base stations for wireless communications, which include transceivers, associated electronic devices for modulation/demodulation, and switches and ports to connect to a backbone network for backhaul communication such as in the case of packet-switched communications.

### II. Methods for Creating a Plurality of Instances

In some embodiments, the inventive methods comprise populating at least the first digital file 20 with a plurality of instances (e.g., 22, 24, 26) comprising data derived from a plurality of gene expression profiling experiments, wherein one or more of the experiments comprise exposing cells to at least one perturbagen. For simplicity of discussion, the gene expression profiling discussed hereafter will be in the context of a microarray experiment.

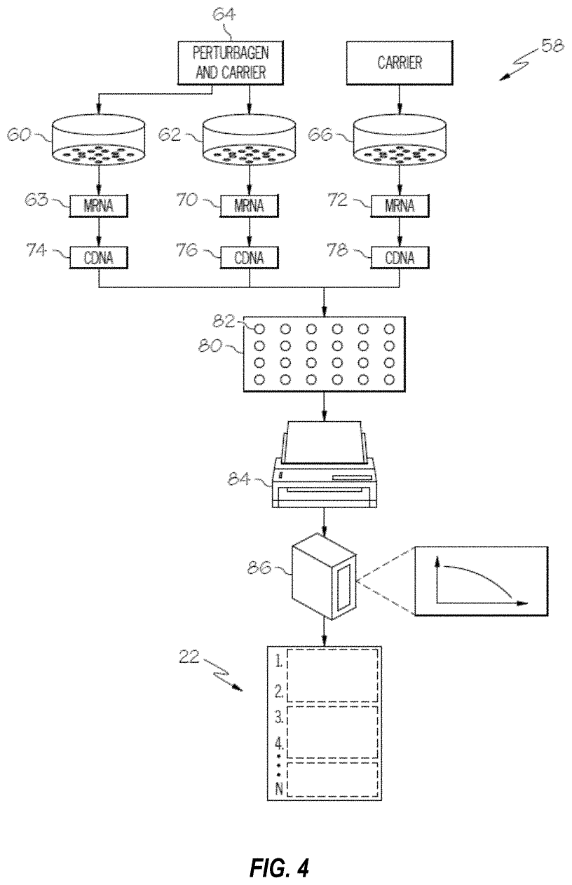

Referring to FIG. 4, one embodiment of the inventive method is illustrated. The method 58 comprises exposing cells 60 and/or cells 62 to a perturbagen 64. After exposure, mRNA is extracted from the cells exposed to the perturbagen. Optionally, mRNA is extracted from reference cells 66 (e.g., control cells) not exposed to the perturbagen for comparison. The mRNA 68, 70, 72 may be reverse transcribed to cDNA 64, 76, 78 and marked with different fluorescent dyes (e.g., red and green) if a two color microarray analysis is to be performed. Alternatively, the samples may be prepped for a one color microarray analysis. A plurality of replicates may be processed if desired. The cDNA samples may be co-hybridized to a microarray 80 comprising a plurality of probes 81. The microarray may comprise thousands of probes 81. In some embodiments, there are between 10,000 and 50,000 gene probes 81 present on the microarray 80. The microarray 80 is scanned by a scanner 83, which excites the dyes and measures the amount of fluorescence. A computing device 85 is used to analyze the raw images to determine the amount of cDNA (or mRNA) in the sample, which is representative of gene expression levels in the cells 60, 62, which is compared to gene expression levels observed in the reference cells 66. The scanner 83 may incorporate the functionality of the computing device 85. The expression levels include: i) up-regulation (e.g., more mRNA or cDNA is present in test material compared to reference material, resulting in more test material (e.g., cDNA 74, 76) being bound by probes compared to the amount of reference material (e.g., cDNA 78) bound to probes), or ii) down-regulation (e.g., more reference material (e.g., cDNA 78) is bound to the probes compared to the amount of test material (e.g., cDNA 74, 76) bound to probes), iii) no differential expression (e.g., similar amounts of the reference material (e.g., cDNA 78) and the test material (e.g., cDNA 74. 76) are bound by the probes), and iv) no detectable signal or noise. The up- and down-regulated genes are referred to as “differentially expressed.”

Microarrays and microarray analysis techniques are well known in the art, and it is contemplated that microarray techniques other than those exemplified herein are suitable for use in the methods, devices and systems of the invention. Any suitable commercial or non-commercial microarray technology and associated techniques may used, such as Affymetrix GeneChip® technology and Illumina BeadChip™ technology. One of skill in the art will appreciate that the invention is not limited to the methodology of the exemplified embodiments and that other methods and techniques are also contemplated to be within the scope of the invention.

Alternately, the probe IDs may be ordered in a non-sorted listing, or may be rank ordered according to an average expression value over multiple instances. In some embodiments, the probe IDs and expression values are listed in a standard order, e.g., defined by the microarray, and manipulated according to the methods described below. For example, a subset of probe IDs may be selected according to average expression values for all of the instances and/or various calculations and/or analysis performed on the probe IDs of interest. This instance data may also further comprise metadata such as perturbagen identification, perturbagen concentration, cell line or sample source, and microarray identification. In some embodiments, the database comprises at least about 50, 100, 250, 500, or 1000 instances and/or less than about 50,000, 20,000, 15,000, 10,000, 7,500, 5,000, or 2,500 instances. Replicates of an instance may created, and the same perturbagen may be used to derive a first instance from a first type of cell and a second instance from a second type of cell and a third instance from a third type of cell.

### III. Signature Free Methods for Querying Perturbagens

A significant challenge to using large probe sets in a query is the presence of batch effect in the C-Map database. Batch effect is a pervasive problem in large-scale data collection efforts that can significantly skew analysis toward identifying batch-based artifacts instead of relevant biological activity. Specifically, replicate samples of a perturbagen-treated cell, a control cell, or a condition-exposed cell may be generated under slightly varying conditions, causing slight differences in measurements taken during profiling experiments. Some factors that have been observed as causing batch effects in microarray experiments include batch of amplification reagent used, time of day when an assay is executed, and even the atmospheric ozone level (Fare et al. 2003). Thus, samples processed and run in different batches often contain systematic non-biological variation that can cause different perturbagens or conditions tested in the same experimental batch to appear closer to one another in structure or mechanism of action than identical perturbagens or conditions tested in different experimental batches. Similarly, batch effect variances can cause similar perturbagens or conditions to appear artificially distinct.

Generally speaking, the technical approach embodied by the signature-free query methods described herein analyzes data such as the gene expression profiles found in a C-Map database. If not already normalized, the data are normalized by applying one of a variety of normalization techniques generally known. By way of example, and without limitation, in some embodiments, the normalization technique employed is a MASS algorithm or a robust multi-array average (RMA) algorithm. The output of the normalization should include an expression value for each probe analyzed in the gene expression profiling experiment. Thus, in some embodiments, an existing C-Map database will include normalized data. In other embodiments, one or more gene expression profiling experiments may be performed, and the data normalized to produce a number of instances (i.e., data from the gene expression profiling experiments). Each instance may include expression value data for all of the probes analyzed in the experiments. The instances may include control instances, test instances, and/or condition instances.

The instances may be further processed to determine a subset of probes to use in the analysis. For each probe, the expression value is averaged over all of the perturbagen and control instances, and the average expression values are sorted. A subset of probes is selected accordingly. In some embodiments, the subset of probes may include the 5,000-10,000 probes with the highest average expression values. In other embodiments, the subset of probes may include more or fewer probes, including all of the probes (i.e., the subset may be the entire set). The subset of probes, in some embodiments, may be selected according to the probes that have average expression values higher than a predetermined threshold. In some embodiments, the expression values may be log transformed before any further processing takes place. In other embodiments, further processing is performed on the raw normalized expression values. In any event, for each control instance in a particular batch, an average expression value for each probe is calculated. For each test instance in the batch, a difference is found between the average expression value for the probe and the expression value for the probe in the test instance. All of the test instances from all of the batches are combined into a single data matrix.

The data matrix is analyzed using multivariate statistical analysis. Though described herein with reference to regularized Fisher Discriminant Analysis using a kernel version of the projection matrix, those of ordinary skill in the art will readily appreciate that other forms of multivariate statistical analysis may be employed in other embodiments. By way of example, and without limitation, a non-kernel version of a projection matrix, a non-regularized Fisher Discriminant Analysis, a Linear Discriminant Analysis, or Generalized Linear Discriminant Analysis could be employed. In any event, the data matrix is reduced by removing non-replicated instances (e.g., instances for perturbagens having only a single genetic expression profile). A projection matrix (or function) is learned using the multivariate statistical analysis, and the entire data matrix (i.e., not the reduced matrix), is projected onto the projection space using the projection matrix (or function). (When using a Kernel version of Fisher Discriminant Analysis, the result is a projection function that utilizes the kernel function to compute the projection. The resulting matrix has a significantly reduced dimension. Similarly to principal component analysis, less significant dimensions can be further dropped to improve the performance of the resulting matrix. The parameters for the regularized Fisher Discriminant Analysis and the number of dimensions to keep for the final projected matrix are determined by cross-validation.

The resulting matrix can be used to determine similarity or dissimilarity between perturbagens. Specifically, a perturbagen in the new matrix may be selected, and the distance in the projected space between the selected perturbagen and every other perturbagen may be calculated using either cosine distance or Euclidean distance. Each of the perturbagens may then be ranked according to its distance from the selected perturbagen. The resulting matrix may also be used to compute a similarity (distance) matrix among all the perturbagens tested. A clustering method can be used to group similar chemicals into groups or organize them into a tree like structure.

Alternately, an average condition profile may be determined and used as a query against the perturbagen data. The gene expression profiles for the condition may be normalized as described above with respect to the gene expression profiles for the perturbagens. The normalized gene expression profiles for the condition (e.g., stored as condition instances) may be averaged to determine an average condition profile by finding the average expression value for each of the subset of probes used to learn the projection matrix. Likewise, the normalized gene expression profiles for the corresponding control instances may be determined in the same manner, and the difference found, for each probe, between the average expression value for the probe in the control instances, and the average expression value for the probe in the condition instances. The vector that results, which may be referred to as an average condition profile, may be projected onto the projection space using the projection matrix. The distance in the projection space between the average condition profile and each of the perturbagens may be calculated using either cosine distance or Euclidean distance. Each of the perturbagens may then be ranked according to their distance from the average condition profile.

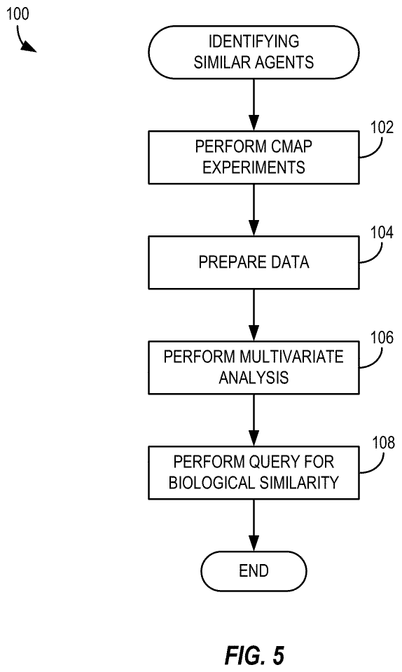

With reference now to FIGS. 5 to 13, computer-implemented methods are described for signature-free identification of biological agents. The presently described methods mitigate the batch effect, allowing a large number of probe sets to be analyzed even when the corresponding samples were processed and run in different experimental batches. The described methods, or portions thereof, may be embodied as instructions stored on one or more computer-readable media.

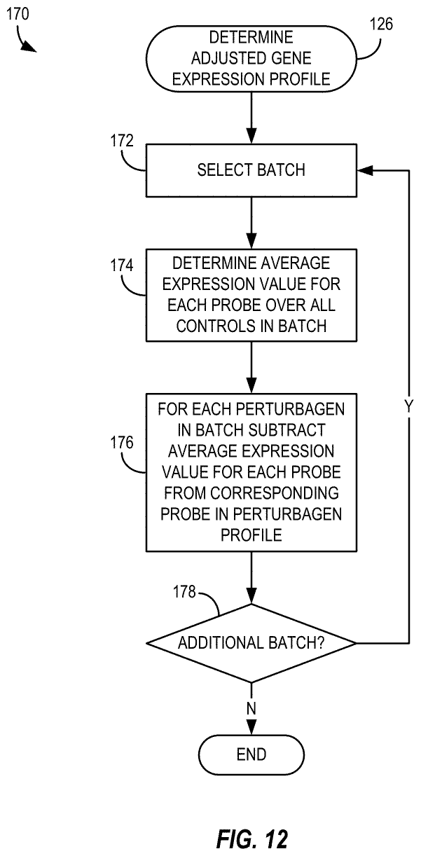

Referring briefly to FIG. 13, tables 160 and 162, which may correspond, for example with data in the data structure of the file 20, each depict a plurality of instances 164 associated with a respective batch. Each of the tables 160, 162 includes, respectively, Y and Z instances 164, and each instance 164 includes expression values 166 for each of N probe IDs 168, where the value N is, in some embodiments, equal to the total number of probes on the microarray. In some embodiments, the data structure 160, 162 may be stored as a set of delimited values. For example, a first value 170 in the data structure 160, 162 is an index “0”, and the following N values 168 identify, respectively, the N probe IDs 168 associated with each of the corresponding expression values 166 of the Y or Z instances 164. Each instance 164 in the data structures 160, 162 includes the expression value 166 for each of the N probes IDs 168. Each batch and, therefore, each data structure may contain control instances 172 (e.g., instances 1A, 2A, 1B, 2B), condition instances 174 (e.g., instances 3A-10A, instances 3B-10B), and test instances 176 (e.g., instances 11A-YA, 11B-ZB).

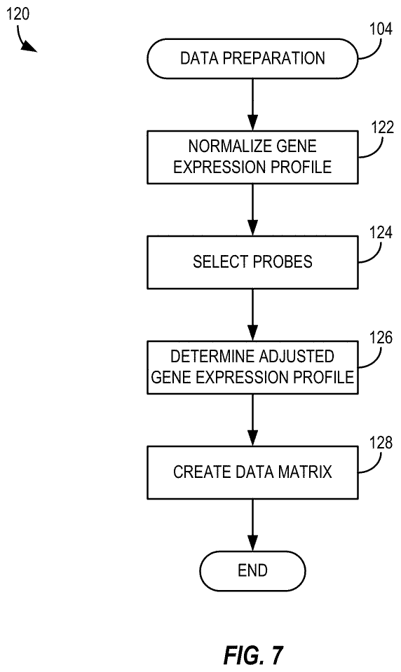

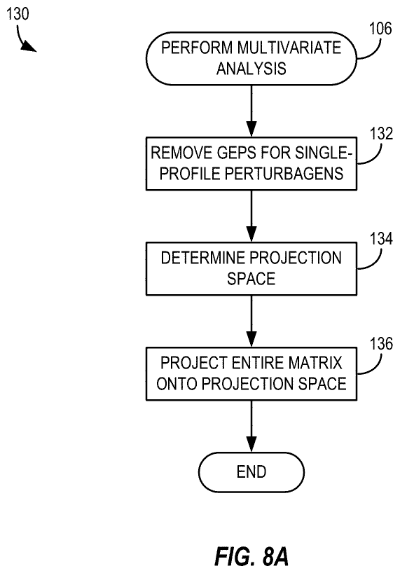

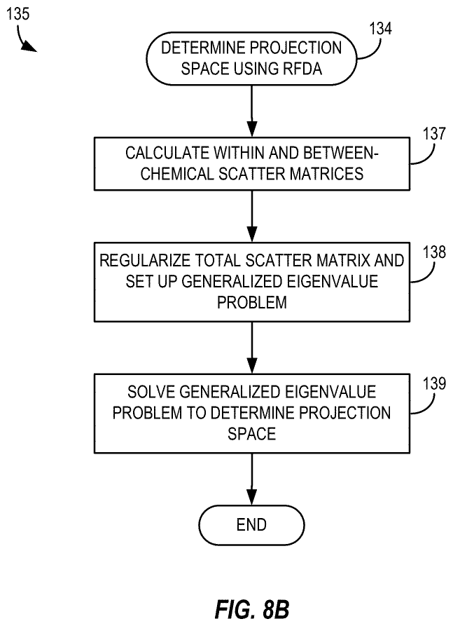

FIG. 5 depicts a method 100 for identifying biological agents that are similar to a query agent. In the method 100, gene expression profiling experiments are performed as described above (block 102). In some embodiments, the gene expression profiling experiments include multiple batches, and each batch includes perturbagen treated cells and control cells. In other embodiments, the gene expression profiling experiments include multiple batches, and each batch includes perturbagen treated cells, control cells, and cells exposed to a condition (e.g., as in the batches corresponding to the tables 160 and 162 in FIG. 13). In still other embodiments, the gene expression profiling experiments include one or more batches that include cells exposed to a condition and one or more batches that do not include cells exposed to a condition. In still other embodiments, one or more of the batches may not include any perturbagen treated cells. The data resulting from the gene expression profiling experiments is then prepared (block 104) as described briefly above and in more detail below (with respect to FIG. 7). The method further includes performing a multivariate analysis (block 106) (described below with respect to FIGS. 8A and 8B). Following the multivariate analysis, one of the gene expression profiles, a query agent, is submitted as a query against the analyzed data to find agents that are similar to the query agent (block 108), as described below with reference to FIG. 9.

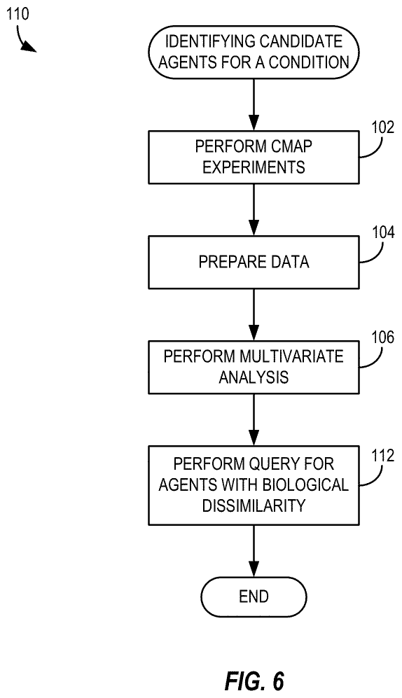

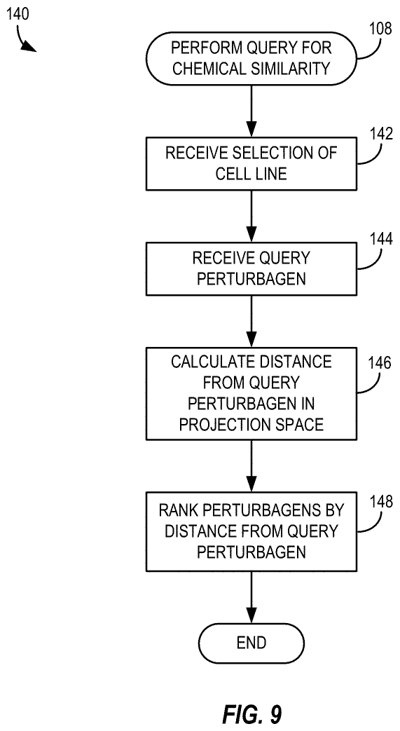

Similarly, FIG. 6 depicts a method 110 for identifying biological agents that are candidates for treating a query condition. In the method 110 gene expression profiling experiments are performed as described above (block 102). The gene expression profiling experiments produce data related to at least control cells, perturbagen treated cells, and cells exposed to the query condition. In some embodiments, the gene expression profiling experiments include multiple batches, and each batch includes perturbagen treated cells and control cells. In other embodiments, the gene expression profiling experiments include multiple batches, and each batch includes perturbagen treated cells, control cells, and cells exposed to a condition. In some embodiments, the gene expression profiling experiments include one or more batches that include cells exposed to a condition and one or more batches that do not include cells exposed to a condition. In some embodiments, one or more of the batches may not include any perturbagen treated cells. The data resulting from the gene expression profiling experiments is then prepared (block 104) as described briefly above and in more detail below (with respect to FIG. 7). The method further includes performing a multivariate analysis (block 106) (described below with respect to FIGS. 8A and 8B). Following the multivariate analysis, an average gene expression profile for a query condition is submitted as a query against the analyzed perturbagen data to find agents most likely to reverse the condition, for example, by identifying agents associated with gene expression profiles most distant (and therefore most dissimilar) from the gene expression profile of the query condition (block 112), as described below with reference to FIG. 10.

Turning now to FIG. 7, a method 120 for data preparation is depicted, corresponding to an embodiment of the data preparation in the methods 100 and 110 (i.e., corresponding to an embodiment of the block 104). In the method 120, each gene expression profile is normalized (block 122) using an expression normalization technique as generally known. In some embodiments, the normalization technique employed is the MASS algorithm. In some embodiments, the normalization technique employed is the RMA technique. In various embodiments, normalization includes finding, for each probe in the gene expression profile, the log of the expression value for the probe.

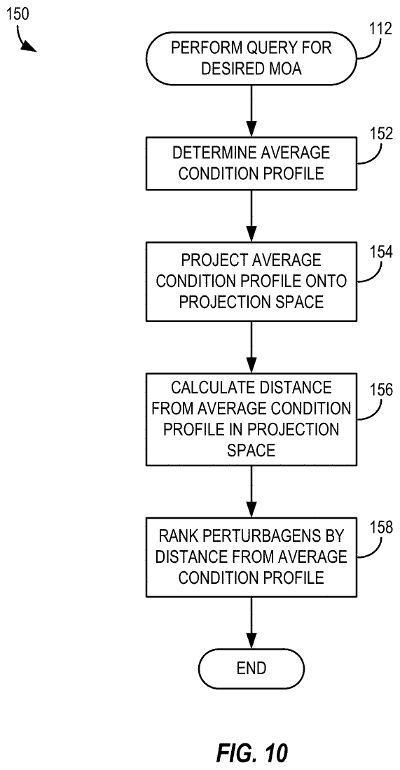

The method 120 continues, in some embodiments, with the selection of probes for further analysis (block 124). FIG. 11 depicts a method 160 for selecting probes, corresponding to the selection of probes (block 124) in the data preparation method 120. With reference to FIGS. 11 and 13, for each of the N probes used to generate the gene expression profiles (i.e., in the instances 164) the expression value 166 is averaged over all of the instances 164 to be analyzed (block 162). That is, if each of 100 (e.g., Y+Z) instances 164 includes expression values 166 for each of 1000 probes, an averaged expression value for each of the 1000 probes is determined. For example, referring to FIG. 13, in an embodiment, the averaged expression value for probe ID1 may be calculated by averaging the expression values 166 for probe ID1 in each of instances 11A-YA and 11B-ZB, the averaged expression value for probe ID2 may be calculated by averaging the expression values 166 for probe ID2 in each of instances 11A-YA and 11B-ZB, etc. The averaged expression values may be sorted and/or ranked. A subset of probes may be selected according to which probes are, on average, most highly expressed (block 166). The subset of probes may be all of the probes (e.g., probe IDs ID1 to IDX) in some embodiments. In some embodiments, the subset of probes may be 5,000 to 10,000 probes. The subset may, in various embodiments include: between about 5,000 probes and about 15,000 probes; between about 5,000 probes and about 25,000 probes; between about 10,000 probes and about 20,000 probes; between about 10,000 probes and about 25,000 probes; between about 25,000 probes about 50,000 probes; more than 10,000 probes; more than 25,000 probes; more than 50,000 probes, etc. In some embodiments, the subset of probes may be selected according to which of the probes has an average expression value higher than a predetermined threshold value.

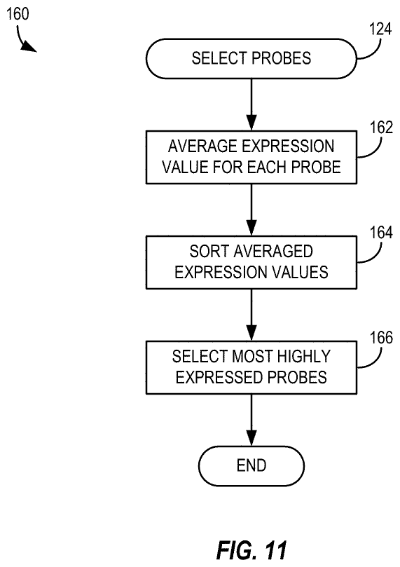

Referring again to FIG. 7, after the probes are selected (block 124), an adjusted gene expression profile is determined for each instance (block 126), as depicted in greater detail in a method 170 of FIG. 12. The method 170 is performed for each of the batches included in the analysis. A batch (e.g., the batch having data in data structure 160) is selected (block 172), and the average expression value for each probe (or each probe in the subset, in embodiments in which a subset of the probes is selected) is calculated over all of the control instances in the selected batch (block 174). Together, the average expression values for the probes over all of the control instances make up an average control gene expression profile. For example, with reference to the data in the data structure 160, an average expression value may be calculated for each of the X probe IDs over the control instances (e.g., instances 1A and 1B). The average expression value for probe ID1 in the batch depicted in data structure 160 would be:

(CNT1_{1A}+CNT1_{2A})/2

where:

CNT1_{1A} is the expression value CNT1 for instance 1A, and

CNT1_{2A} is the expression value CNT1 for instance 2A;

for probe ID2 would be:

(CNT2_{1A}+CNT2_{2A})/2

where:

CNT2_{1A} is the expression value CNT2 for instance 1A, and

CNT2_{2A} is the expression value CNT2 for instance 2A; etc.

Next, a differential expression value (also referred to herein as an “adjusted test gene expression profile” or an “adjusted gene expression profile”) is determined for each perturbagen instance in the batch by determining the difference between the average expression value for each probe (or each probe in the subset) and the expression value 166 for the corresponding probe in the perturbagen instance (e.g., the instances 11A-YA, 11B-ZB) (block 176). Continuing the previous example, the differential expression value for probe ID1 of instance 11A would be:

CNT1_{11A}−[(CNT1_{1A}+CNT1_{2A})/2];

the differential expression value for probe ID2 of instance 11A would be:

CNT2_{11A}−[(CNT2_{1A}+CNT2_{2A})/2];

the differential expression value for probe ID1 of instance 12A would be:

CNT1_{12A}−[(CNT1_{1A}+CNT1_{2A})/2]; etc.

If there is an additional batch (e.g., the batch depicted in the data structure 162) (block 178), control returns to selecting the next batch (block 172) and the method 170 is re-executed until the method 170 is performed for all batches to be analyzed. The adjusted gene expression profiles, which, for each instance, include all of the differential expression values, is combined into a data matrix (block 128, FIG. 7). This data matrix will be referred to hereafter as a data matrix or a perturbagen data matrix, though it should be clear that the data matrix may include instance data for perturbagen-treated cells, condition-exposed cells, etc. The perturbagen data matrix may be stored in, for example, the computer-readable medium 16 and/or the computer-readable medium 38.

In both the method 100 and the method 110, performing the multivariate analysis (block 106) involves, in some embodiments, the execution of a method 130, depicted in FIG. 8A. For the purpose of learning the projection matrix, instances for perturbagens with only a single gene expression profile are removed from the perturbagen data matrix to create a reduced perturbagen data matrix (block 132) (sometimes referred to simply as a “reduced data matrix”), which may also be stored on one or both of the computer-readable mediums 16, 38. The projection matrix is learned according to a method of multivariate statistical analysis using the reduced perturbagen data matrix and, in particular, may be learned using a regularized Fisher Discriminant analysis (block 134). In a method 135, depicted in FIG. 8B, for instance, the projection space is determined (block 134) using regularized Fisher discriminant analysis (RFDA). The within- and between-chemical scatter matrices are calculated (block 137). The total scatter matrix is regularized and a generalized eigenvalue problem set up (block 138). The generalized eigenvalue problem is solved to determine the projection space (block 139). In some embodiments, the projection matrix may be a RBF kernel projection matrix, as described in Z. Zhang et al. “Regularized Discriminant Analysis, Ridge Regression and Beyond”; Journal of Machine Learning Research 11 (2010) 2199-2228, August 2010). The entire matrix (i.e., the perturbagen data matrix created at block 128) is then projected onto the projection space using the projection matrix, creating a projection space matrix with significantly reduced dimension (block 136). Similar to the other matrices described herein, the projection space matrix may be stored on one or both of the computer-readable mediums 16, 38.

Using the projection space matrix, it is possible to determine the similarity (or difference) between gene expression profiles in the projection space. The methods 100 and 110, for example, perform queries for similar biological activity (block 108) and biological dissimilarity (i.e., agents most likely to reverse a clinical endpoint) (block 112), respectively, by looking at the distances between instances depicted in the projection space matrix. Turning first to the method 100, FIG. 9 depicts a method 140 for performing a query for similar biological activity between instances mapping to two points in the projection space (e.g., for performing a query for similar activity between perturbagens) (block 108). The method includes, in some embodiments, receiving a selection of the cell line to analyze (block 142). For example, a user may select a first cell line (e.g., tert keratinocytes) on which a number of perturbagens have been tested, or may select a second cell line (e.g., BJ Fibroblasts) on which a number of perturbagens have been tested. The same or different set of perturbagens may have been tested on each of the first and second cell lines. Additionally, in some embodiments, the method may include receiving a selection related to treatment of replicated instances. That is, each chemical instance (i.e., including each replicate of each perturbagen gene expression profile) may be examined in the projection space, or instances of chemical replicates may be averaged. Averaging of chemical replicates may occur before or after projection into the projection space matrix, in different embodiments.

A query perturbagen (also referred to as a query agent) is then selected from the perturbagens in the projection space matrix (block 144). Of course, while described here as a query “perturbagen,” the query agent could be any vector in the projection space matrix, including a vector for a perturbagen, a vector for a hypothetical chemical structure, a vector corresponding to the gene profile for a condition-exposed cell, etc. The distance from the query perturbagen in the projection space is calculated for each instance (or for a selected subset of instances) in the projection space matrix (block 146). In some embodiments, the distance is calculated as a cosine distance. In some embodiments, the distance is calculated as a Euclidean distance. In any event, the various perturbagens (or other data) in the projection space matrix are ranked according to the distance of each from the query perturbagen (block 148). The perturbagens closest to (i.e., having the shortest distance from) the query perturbagen in the projection space induce a gene expression profile that is the most similar to that of the query perturbagen. Methods, other than ranking, for determining relative distances between the query perturbagen and other instances in the projection space may be used in some embodiments.

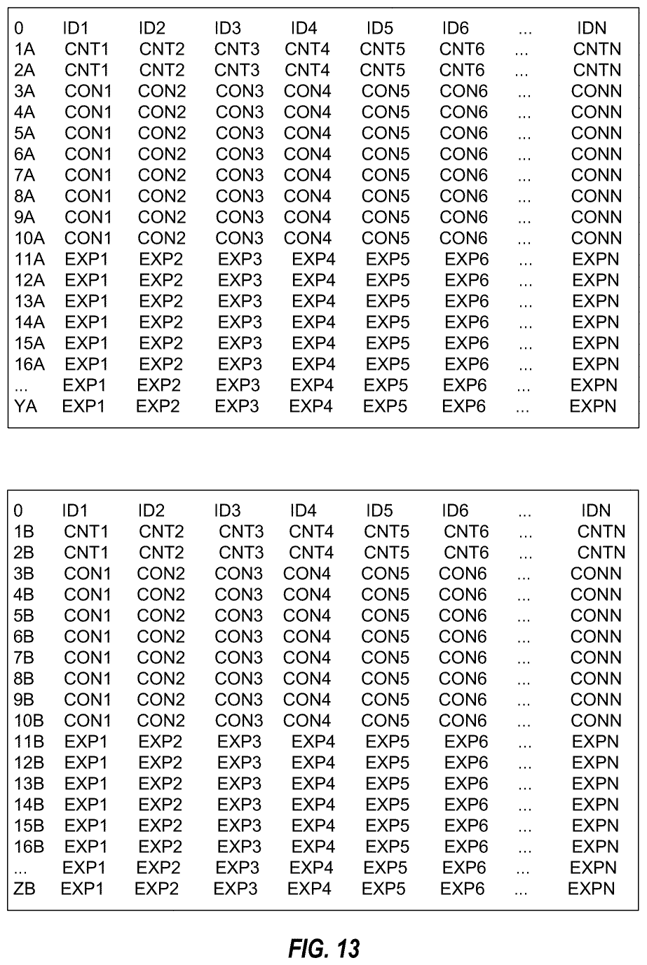

FIG. 14 illustrates the results 180 of an exemplary query having a query perturbagen 182. As illustrated (and as expected), the query perturbagen 182 has a distance 184 of 0.0 from itself. The results 180 also indicate, in the depicted example, a Chip ID 186 and a corresponding chemical name 188. The exemplary results illustrate that replicates of the same chemical (o-phenanthroline) (e.g., chemicals ranking 2 and 3) have the smallest distance from the query perturbagen. The perturbagen holding ranks 4 and 5 in the results 180 is 2,6-Di(2-pyridyl)pyridine. As depicted, the chemical structure 187 of o-phenanthroline is similar to the chemical structure 189A of 2,6-Di(2-pyridyl)pyridine. The chemical structures 189B and 189C of 4,4′-Dimethyl-2,2′-bipyridine and 3,4,7,8-Tetramethylphenanthroline, respectively, are slightly less similar to that of o-phenanthroline and are ranked 6-7 and 8-9, respectively, according to distance from o-phenanthroline.

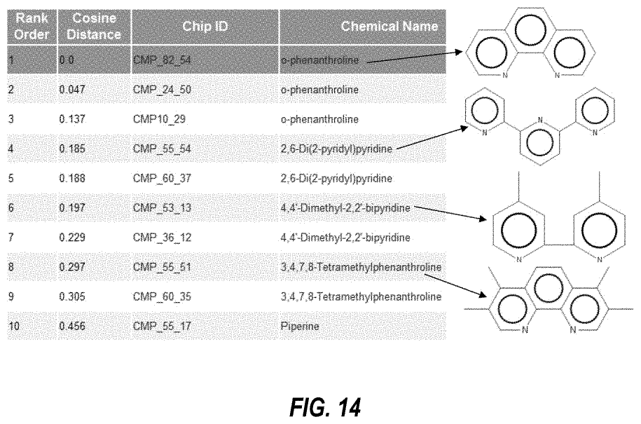

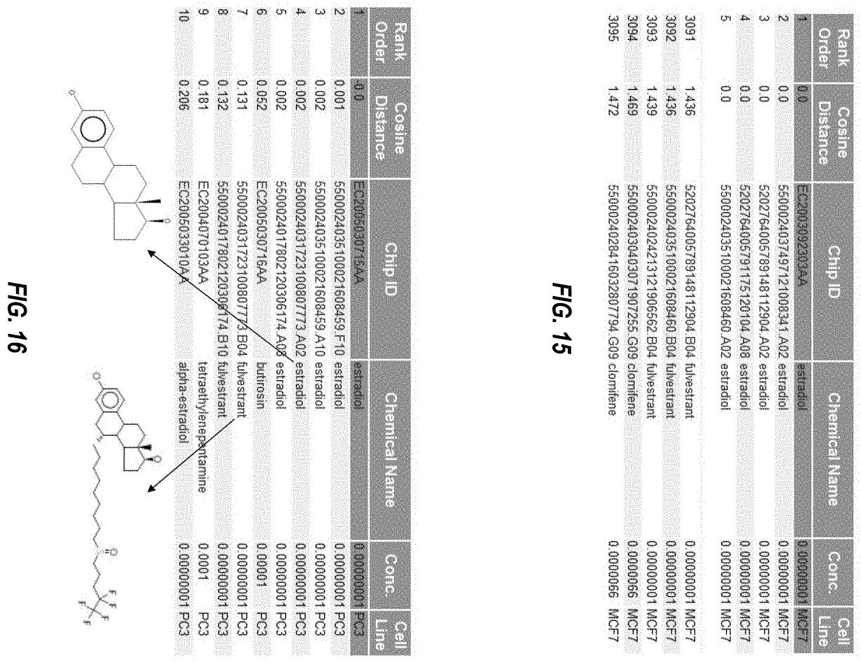

With reference to FIGS. 15 and 16, the effect of different perturbagens on different cell types at the transcriptional level is readily apparent. In FIG. 15, a table 200 depicts the top five and bottom five chemicals ranked according to distance 202 from a query perturbagen 204 (estradiol) in a cell line MCF7 206. Among the top five most similar chemical instances 208, are Estradiol replicates. At the opposite end (most dissimilar) are anti-estrogenic agents Clomifene and Fulvestrant 210. This behavior is consistent with the fact that MCF7 cell line expresses estrogen receptors and the top and bottom listed chemicals 208, 210, respectively, act as agonists and antagonists. However, as shown in FIG. 16, a table 212 depicting the top 10 chemicals ranked according to distance 214 from the same query perturbagen 216 (estradiol) in a different cell line PC3 218, shows that when looking at Estradiol treatments in PC3 (prostate cancer) cells, which lack estrogen receptors, Fulvestrant is found to be similar to Estradiol. The structures 220, 222 of Estradiol and Fulvestrant are similar, and the agents induce a similar transcriptional response in the pC3 cell line lacking estrogen receptors. These results validate the ability of the methods, systems, and apparatus described herein to extract meaningful signal from noisy gene expression data even in the presence of a mechanism of action that is dependent on the cell line in question.

Turning next to the method 110, FIG. 10 depicts a method 150 for performing a query for perturbagens eliciting a biological response that is dissimilar to that induced by a condition (e.g., chemicals likely to reverse a particular condition in a cell) (block 112). The method includes determining an average condition profile to use as a query (block 152), as described above. Specifically, the average condition profile (also referred to as an “adjusted condition gene expression profile”) may be calculated by finding the average expression value for each of the subset of probes used to learn the expression matrix. That is, if all of probes ID1-IDN (referring to FIG. 13) were used to learn the expression matrix, the average expression profile for a condition tested in instances 3A-10A and 3B-10B would include an average expression value for probe ID1:

(CON1_{3A}+CON1_{. . . A}+CON1_{10A}+CON1_{3B}+CON1_{. . . B}+CON1_{10B})/16;

an average expression value for probe ID2:

(CON2_{3A}+CON2_{. . . A}+CON2_{10A}+CON2_{3B}+CON2_{. . . B}+CON2_{10B})/16;

etc. Of course, this assumes that each of instances 3A-10A and 3B-10B is for the cells exhibiting the same condition, which need not necessarily be the case. The average control profiles for the condition of interest are subtracted from the average condition profile, as described above.

The average condition profile is projected onto the projection space (block 154). The distance from the average condition profile to each of the perturbagens in the projection space matrix is determined (block 156) and, at least in some embodiments, the perturbagens are ranked according to the distance of each in the projection space from the average condition profile (block 158). In some embodiments, the distance is calculated as a cosine distance. In some embodiments, the distance is calculated as a Euclidean distance. The perturbagens further (i.e., having the greatest distance) in the projection space from the average condition profile used as the query are the most likely to reverse the expression pattern of the average condition profile.

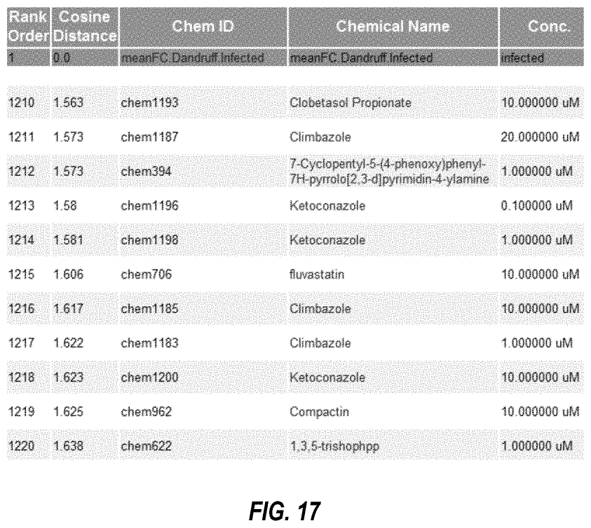

FIG. 17 is a table 230 of results 232 corresponding to chemical instances that reverse (or mimic) a clinical outcome. A query condition 234 (e.g., dandruff) corresponds to an average condition profile for condition-treated cells. The rankings of perturbagens, including Climbazole and Ketocanozole, as more distant from the query condition 234 indicates the perturbagens' potential usefulness for treating the query condition. Specifically, Climazole and Ketocanozole are well-known anti-dandruff agents. Similarly, if gene expression data for any condition of interest (and associated control data) are available, the data can be analyzed using the methods, systems, and apparatus described herein to perform signature-free queries that identify treatments that best mimic or reverse the differential gene expression pattern associated with a condition.

While the methods and systems above are described with respect to analysis of gene expression profile data, it will be appreciated that the methods could readily be applied in the analysis of data sets other than gene expression profile data including, by way of example and not limitation, data sets related to other biomarkers.

Every document cited herein is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent any meaning or definition of a term in this document conflicts with any meaning or definition of the same term in a document incorporated by reference, the meaning or definition assigned to that term in this document shall govern.

The values disclosed herein are not to be understood as being strictly limited to the exact numerical values recited. Instead, unless otherwise specified, each such value is intended to mean both the recited value and a functionally equivalent range surrounding that value.

The invention should not be considered limited to the specific examples described herein, but rather should be understood to cover all aspects of the invention. Various modifications, equivalent processes, as well as numerous structures and devices to which the invention may be applicable will be readily apparent to those of skill in the art. Those skilled in the art will understand that various changes may be made without departing from the scope of the invention, which is not to be considered limited to what is described in the specification.

What is claimed is:

1. A method for formulating a composition by identifying similarities between gene expression profiles of cells exposed to different perturbagens, the method comprising:
accessing data related to gene expression profile (GEP) experiments for a plurality of batches, each batch associated with a plurality of control instances and a plurality of test instances, each of the plurality of control instances including information related to a GEP for a control cell and each of the plurality of test instances including information related to a cell exposed to a corresponding perturbagen, each of the instances including an expression value for each of a plurality of probes;
determining, for each batch, an average control GEP for the batch, the average control GEP for the batch determined by averaging expression values for each of a subset of probes over all of the control GEPs;
determining an adjusted test GEP for each test instance in a batch, each adjusted test GEP determined by subtracting the expression values for each of the subset of probes in the test instance from the expression value of the average control GEP for the corresponding batch;
creating a data matrix by combining all of the adjusted test GEPs from all of the plurality of batches;
creating a reduced data matrix by removing from the data matrix adjusted test GEPs for any perturbagen for which there exists in the data matrix only a single adjusted test GEP;
performing a multivariate statistical analysis on the reduced data matrix to create a projection matrix or a projection function defining a projection space;
projecting the data matrix onto the projection space using the projection matrix or the projection function to create a projected matrix;
determining a number of dimensions to keep for the projected matrix;
comparing the positions of the adjusted test GEPs in the projection space to identify perturbagens with similar biological activity; and
formulating a composition comprising an acceptable carrier and at least one perturbagen selected according to its proximity in the projection space to a second perturbagen.

2. A method according to claim 1, wherein comparing the position of the adjusted test GEPs in the projection space comprises:
receiving a selection of an adjusted test GEP corresponding to a query perturbagen; and
calculating a distance in the projection space from the adjusted test GEP corresponding to the query perturbagen to each of the adjusted test GEPs in the data matrix.

3. A method according to claim 2, wherein calculating a distance in the projection space comprises calculating a Euclidian distance.

4. A method according to claim 2, wherein calculating a distance in the projection space comprises calculating a cosine distance.

5. A method according to claim 2, wherein comparing the position of the adjusted test GEPs in the projection space further comprises:
ranking the perturbagens according to the distance in the projection space from the adjusted test GEP corresponding to the query perturbagen to the adjusted test GEP corresponding to the perturbagen to be ranked.

6. A method according to claim 1, wherein the selected subset of probes is determined by a method comprising:
determining an average expression value for each probe over the plurality of control and test instances;
sorting the average expression values; and
selecting a number of the most highly expressed probes.

7. A method according to claim 1, further comprising extracting a plurality of biological samples from a respective plurality of cells treated with perturbagens and subjecting the biological samples to microarray analysis.

8. A method for formulating a composition by identifying differences between gene expression profiles of cells exposed to a perturbagen and gene expression profiles of cells exposed to a condition, the method comprising:
accessing data related to gene expression profile (GEP) experiments for a plurality of batches, each batch associated with a plurality of test instances associated with a perturbagen and a plurality of control instances, each of the instances including an expression value for each of a plurality of probes;
determining, for each batch, an average control GEP for the batch, the average control GEP for the batch determined by averaging the expression values for each of a subset of probes over all of the control instances;
determining an adjusted test GEP for each test instance in a batch, each adjusted test GEP determined by subtracting the expression values for each of the subset of probes in the test instance from the expression value for the corresponding probe in the average control GEP for the corresponding batch;
creating a data matrix by combining all of the adjusted test GEPs from all of the plurality of batches;
creating a reduced data matrix by removing from the data matrix adjusted test GEPs for any perturbagen for which there exists in the data matrix only a single adjusted test GEP;
performing a multivariate statistical analysis on the reduced data matrix to create a projection matrix or a projection function defining a projection space;
projecting the data matrix onto the projection space using the projection matrix or the projection function to create a projected matrix;
determining a number of dimensions to keep for the projected matrix;
determining an adjusted condition GEP;
projecting the adjusted condition GEP onto the projection space using the projection matrix;
comparing the position of the adjusted condition GEP in the projection space to the positions of the adjusted test GEPs in the projection space to identify one or more perturbagens; and
formulating a composition comprising an acceptable carrier and at least one perturbagen selected according to the comparison of the positions.

9. A method according to claim 8, wherein determining an adjusted condition GEP comprises:
determining a second average control GEP for a second batch, the second batch including GEPs for control cells and GEPs for cells exposed to the condition;
determining an average condition GEP for the second batch; and
determining the adjusted condition GEP by determining, for each of the subset of probes, the difference between the expression value for the probe in the second average control GEP and the expression value for the probe in the average condition GEP.

10. A method according to claim 9, wherein determining an average condition GEP for the second batch comprises determining, for each of the subset of probes, an average expression value for the probe over a plurality of condition GEPs.

11. A method according to claim 8, wherein comparing the position of the adjusted condition GEP in the projection space to the positions of the adjusted test GEPs in the projection space to identify one or more perturbagens comprises:
calculating a distance in the projection space from the average condition profile to each of the adjusted test GEPs in the data matrix.

12. A method according to claim 11, wherein calculating a distance in the projection space comprises calculating a Euclidian distance.

13. A method according to claim 11, wherein calculating a distance in the projection space comprises calculating a cosine distance.

14. A method according to claim 11, wherein comparing the position of the adjusted condition GEP in the projection space to the positions of the adjusted test GEPs in the projection space to identify one or more perturbagens further comprises:
ranking the one or more perturbagens according to the distance in the projection space from the average condition profile to the adjusted test GEP for each perturbagen.

15. A method according to claim 8, wherein the selected subset of probes is determined by a method comprising:
determining an average expression value for each probe over the plurality of control and test instances;
sorting the average expression values; and
selecting a number of the most highly expressed probes.

16. A method according to claim 8, wherein the selected subset of probes is determined by a method comprising selecting a predetermined number of probes according to relative expression of the probes.

17. A method according to claim 8, wherein the selected subset of probes is determined by a method comprising selecting a subset of probes above a predetermined threshold expression level.

18. A method according to claim 8, wherein performing a multivariate statistical analysis comprises performing a Fisher discriminant analysis.

19. A method according to claim 8, wherein performing a multivariate statistical analysis comprises performing a regularized Fisher discriminant analysis.

20. A method according to claim 8, wherein performing a multivariate statistical analysis comprises performing a kernel discriminant analysis.
